(19) United States
(12) Patent Application Publication
(10) Pub. No.: US 2010/0184867 A1
(43) Pub. Date: Jul. 22, 2010

(54) USE OF ACTIVE AGENTS FOR STIMULATING THE EXPRESSION OF FN3K AND/OR FN3K RP FOR COMBATING AGEING OF THE SKIN

(75) Inventors: Christelle Lasserre, Jersey City, NJ (US); Elena Fedorova, Whippany, NJ (US); Yannick Maestro, Martigues (FR)

Correspondence Address: YOUNG & THOMPSON, 209 Madison Street, Suite 500, Alexandria, VA, 22314, US

(73) Assignee: CHANEL PARFUMS BEAUTE, Neuilly Sur Seine (FR)

(21) Appl. No.: 12/664,124

(22) PCT Filed: May 13, 2008
(86) PCT No.: PCT/EP2008/055861

§ 371 Date: Dec. 11, 2009

Related U.S. Application Data
(60) Provisional application No. 60/943,101, filed on Jun. 11, 2007.

(51) Int. Cl. A61K 8/34; A61Q 19/08

(52) U.S. Cl. 514/724

(57) ABSTRACT

A cosmetic process for caring for human skin, which is intended for preventing and/or combating at least one sign of ageing of the skin or the ‘orange peel’ appearance of the skin, includes the topical application to the skin of a composition containing at least one active agent for stimulating the expression of FN3K and/or FN3K RP. The use of this active agent for preventing and/or combating at least one sign of ageing of the skin or the ‘orange peel’ appearance of the skin associated with cellulite is also disclosed.

The present invention relates to a cosmetic process for caring for human skin, which is intended for preventing or combating the signs of ageing of the skin and/or the “orange peel” appearance of the skin, comprising the topical application to the skin of a composition containing at least one active agent for stimulating the expression of fructosamine-3-kinase or its related protein (FN3K RP).

The skin consists mainly of three layers, namely, starting from the uppermost layer, the epidermis, the dermis and the hypodermis.

The epidermis in particular consists of keratinocytes (predominantly), melanocytes (involved in pigmenting the skin) and Langerhans cells. Its function is to protect the body from the external environment and to ensure its integrity, and especially to halt the penetration of microorganisms or chemical substances, to prevent evaporation of the water contained in the skin and to constitute a barrier against external attack and especially against ultraviolet rays (UV).

The dermis, for its part, comprises an extracellular matrix containing elastin and collagen fibers that give the skin its elasticity and firmness properties. Collagen, and in particular type I collagen, is the main constituent of the dermis. With age, a reduction in its synthesis is observed, and also an increase in the expression of matrix metalloproteases (MMP) involved in its degradation, which are responsible for slackening of the skin, and also crosslinking of collagen leading to an increase in the rigidity of the skin, which thus becomes less tonic. This crosslinking results especially from non-enzymatic glycation, the Maillard reaction, which involves the reaction of a glucose molecule with an amine and in particular a lysine side chain of a protein such as collagen, leading, via the Amadori rearrangement, to a fructosamine such as fructoselysine (FL), which is capable of converting into an amino aldose and of resulting finally in the formation of advanced glycation products (AGEs). Fructoselysine and/or fructoselysine-6-phosphate may be phosphorylated according to a reaction catalyzed by fructosamine-3-kinase (FN3K) and/or FN3K RP (the protein relates to FN3K), which thus acts as a “deglycation” enzyme, by concomitantly forming—according to a cascade reaction—3-deoxyglucosone (3DG) or deoxyglucosone phosphate, which are, however, also liable to glycate the skin proteins and to form advanced glycation products (AGEs).

To combat this phenomenon of formation of 3DG and its consequences on the appearance of the skin, FN3K and/or 3DG inhibitors such as N-methylglucamine or 3-O-methyl sorbitollysine have been proposed (US 2007/065 443, US 2006/110 439, US 2003/219 440).

Other solutions that have been proposed for preventing or reducing the glycation of proteins include the topical administration to the skin of botanical extracts such as extracts of mulberry (WO 01/45648), of olive (JP2001-122758) or of saxifrage (JP2001-131051) or alternatively of synthetic active agents such as thiazole derivatives (US 2005/0 137 239).

In addition to its effects on ageing of the skin, the glycation of proteins is also involved in the “orange peel” appearance of the skin associated with cellulite, which results from the glycation of the collagen constituting the majority of the connective trabeculae, which has the effect of trapping the fat globules and thus of forming on the skin a succession of bumps (formed by fatty lumps) and of hollows (formed by the rigidified connective trabeculae), giving the skin an appearance similar to that of orange peel.

In view of the foregoing text, it would therefore be useful to have available novel cosmetic active agents for preventing or combating aesthetic disorders resulting from the glycation of skin proteins, whether these disorders concern the signs of ageing of the skin or the “orange peel” appearance of the skin.

In addition, given the ever-increasing search by consumers for natural products containing the fewest possible synthetic ingredients, and the increasingly burdensome regulatory constraints on compounds derived from the chemical industry, it would be desirable for these cosmetic active agents to be of plant origin.

Now, the Applicant has, to its credit, demonstrated, unexpectedly, that FN3K and FN3K RP, involved in the deglycation of proteins, are expressed in the epidermis by keratinocytes and fibroblasts. The Applicant has also shown that FN3K in skin diminishes with increasing age and that the absence of FN3K in reconstructed skins had the same consequence as the effect of glycation on catalase expression and on epidermal thickness. The Applicant has moreover, to its credit, developed an appropriate screening test for selecting active agents that act on these biological targets and for identifying plant extracts that satisfy this test, thus making it possible to meet the above-mentioned needs.

This approach is entirely novel, insofar as the above prior art rather suggests treating the signs of ageing of the skin by inhibiting FN3K and/or FN3K RP.

One subject of the present invention is thus a cosmetic process for caring for human skin, which is intended for preventing and/or combating at least one sign of ageing of the skin (such as the formation of wrinkles and fine lines and/or loss of firmness and/or loss of elasticity of the skin), comprising the topical application to the skin of a composition containing at least one active agent for stimulating the expression of fructosamine-3-kinase (referred to hereinbelow as FN3K) and/or its related protein (referred to hereinbelow as FN3K RP).

A subject of the invention is also a cosmetic process for caring for human skin, which is intended for preventing and/or combating the “orange peel” appearance of the skin, comprising the topical application to the skin of a composition containing at least one active agent for stimulating the expression of FN3K and/or FN3K RP.

A subject of the present invention is also the cosmetic use of an active agent for stimulating the expression of FN3K and/or FN3K RP, for preventing and/or combating at least one sign of ageing of the skin and/or for combating the “orange peel” appearance of the skin.

As a preamble, it is pointed out that the expression “active agent for stimulating the expression of FN3K and/or FN3K RP” means a compound or (especially in the case of a botanical extract) a mixture of compounds capable of stimulating the expression of FN3K and/or FN3K RP relative to an untreated control, which is determined in particular by means of the real-time polymerase chain amplification method (RT-PCR) as described in the examples below.

The active agent for stimulating the expression of FN3K and/or FN3K RP may be used in a proportion of from 0.00001% to 10% by weight, preferably in a proportion of from 0.0001% to 5% by weight and more preferably in a proportion of from 0.001% to 1% by weight relative to the total weight of the composition.

The active agents that may be used according to the invention are advantageously botanical extracts, i.e. active agents obtained by extraction, using any type of solvent, of any part of a plant such as bark, wood, roots, rhizomes, stalks, leaves, fruit or flowers, for example.

An example of such active agents especially comprises an alcoholic extract of Butea frondosa blossom. This extract may be obtained by alcoholic extraction using at least one monoalcohol such as ethanol, methanol or isopropanol and/or at least one glycol such as propylene glycol or dipropylene glycol, optionally mixed with water. The extraction is then performed in the absence of any other solvent. In general, in the case of aqueous-alcoholic solvents, it is preferable for the volume ratio of the alcohol to water to be between 70% and 96%.

In general, the extraction may be performed on fresh or dried flowers, optionally chopped or ground, in the usual manner. The extraction is generally performed by immersing or gently shaking the flowers in one or more of the solvents mentioned above at temperatures ranging, for example, from room temperature to 100° C. and advantageously from 30 to 70° C., for a time of about 30 minutes to 12 hours and preferably from 1 to 8 hours. The solution is then preferably filtered so as to remove the insoluble substances of the plant. The solvent is also, where appropriate, removed if it is a volatile solvent, for instance ethanol, methanol or isopropanol. This extraction step is common in the field of plant extracts and a person skilled in the art is capable of adjusting the reaction parameters thereof on the basis of his general knowledge.

After this extraction step, an extract of Butea frondosa flowers is obtained, which may then, according to an advantageous aspect of the invention, be subjected to a decolorizing step, especially using active charcoal in the presence of a solvent. The weight of active charcoal is preferably between 0.5% and 50% of the weight of the extract. One or more solvents chosen from water, C₁-C₄ alcohols such as methanol, ethanol or isopropanol, polar organic solvents such as propylene glycol or dipropylene glycol, or any other solvent that is common in the field, may especially be used. The volatile solvents may then be removed under reduced pressure.

Preferably, for use in the treatment of the signs of ageing of the skin, the active agent used according to the invention or the composition used in the process according to the invention are applied to aged skin, i.e. wrinkled skin and/or skin showing signs of slackening. They may advantageously be applied to the skin of the face, the neck and optionally the neckline.

In addition, when they are intended to be used in the treatment of the “orange peel” appearance of the skin, the active agent used according to the invention or the composition used in the process according to the invention are generally applied to the skin of at least part of the body such as the legs and/the thighs and/or the buttocks and/or the stomach and preferably to skin showing signs of cellulite.

The composition containing this active agent may be applied in the morning and/or in the evening, to the entire face, the neck and optionally the neckline or even the body.

The composition used according to the invention generally comprises, besides the active agent described previously, a physiologically acceptable and preferably cosmetically acceptable medium, i.e. a medium that is suitable for use in contact with human skin without any risk of toxicity, incompatibility, instability or allergic response and especially that does not cause any sensations of discomfort (redness, tautness, stinging, etc.) that are unacceptable to the user.

This medium generally contains water and optionally other solvents such as ethanol.

The composition used according to the invention may be in any form that is suitable for topical application to the skin and in particular in the form of an oil-in-water, water-in-oil or multiple emulsion (W/O/W or O/W/O), which may optionally be microemulsions or nanoemulsions, or in the form of an aqueous dispersion, a solution, an aqueous gel or a powder. It is preferable for this composition to be in the form of an oil-in-water emulsion.

This composition is preferably used as a care and/or cleansing product for facial and/or bodily skin and it may especially be in the form of a fluid, a gel or a mousse, conditioned, for example, in a pump-dispenser bottle, an aerosol or a tube, or in the form of cream conditioned, for example, in a jar. As a variant, it may be in the form of a makeup product and in particular a foundation or a loose or compact powder.

It may contain various adjuvants, such as at least one compound chosen from:
oils, which may be chosen especially from: linear or cyclic, volatile or non-volatile silicone oils, such as polydimethylsiloxanes (dimethicones), polyalkylcyclosiloxanes (cyclomethicones) and polyalkylphenyisiloxanes (phenyl dimethicones); synthetic oils such as fluoro oils, alkylbenzoates and branched hydrocarbons such as polyisobutylene; plant oils and especially soybean oil or jojoba oil; and mineral oils such as liquid petroleum jelly;
waxes such as ozokerite, polyethylene wax, beeswax or carnauba wax;
silicone elastomers obtained especially by reaction, in the presence of a catalyst, of a polysiloxane containing at least one reactive group (especially hydrogen or vinyl) and bearing at least one alkyl group (especially methyl) or phenyl, in a terminal and/or side position, with an organosilicone such as an organohydrogenopolysiloxane;
cyclic dimethicone/vinyl dimethicone copolymers, such as those marketed by JEEN under the trade names Jeesilc® PS (including PS-CM);
surfactants, preferably emulsifying surfactants, whether they are nonionic, anionic, cationic or amphoteric, and in particular fatty acid esters of polyols such as fatty acid esters of glycerol, fatty acid esters of sorbitan, fatty acid esters of polyethylene glycol and fatty acid esters of sucrose; fatty alkyl ethers of polyethylene glycol; alkylpolyglucosides; polysiloxane-modified polyethers; betaine and derivatives thereof; polyquaterniums; ethoxylated fatty alkyl sulfate salts; sulfosuccinates; sarcosinates; alkyl and dialkyl phosphates, and salts thereof; and fatty acid soaps;
co-surfactants such as linear fatty alcohols and in particular cetyl alcohol and stearyl alcohol;
thickeners and/or gelling agents, and in particular crosslinked or non-crosslinked, hydrophilic or amphiphilic homopolymers and copolymers, of acryloylmethylpropanesulfonic acid (AMPS) and/or of acrylamide and/or of acrylic acid and/or of acrylic acid salts or esters; xanthan gum or guar gum; cellulose derivatives; and silicone gums (dimethiconol);
organic screening agents, such as dibenzoylmethane derivatives (including butylmethoxydibenzoyl-methane), cinnamic acid derivatives (including ethylhexyl methoxycinnamate), salicylates, para-aminobenzoic acids, β,β′-diphenyl acrylates, benzophenones, benzylidenecamphor derivatives, phenylbenzimidazoles, triazines, phenyl-benzotriazoles and anthranilic derivatives;
inorganic screening agents, based on mineral oxides in the form of coated or uncoated pigments or nanopigments, and in particular based on titanium dioxide or zinc oxide;
dyes;
preserving agents;
sequestrants such as EDTA salts;
fragrances;
and mixtures thereof, without this list being limiting.

Examples of such adjuvants are especially mentioned in the CTFA dictionary (International Cosmetic Ingredient Dictionary and Handbook published by The Cosmetic, Toiletry and Fragrance Association, 11th edition, 2006), which describes a wide variety, without limitation, of cosmetic and pharmaceutical ingredients usually used in the skincare industry, that are suitable for use as additional ingredients in the compositions according to the present invention.

The composition may also contain at least one compound with an optical effect such as fillers, pigments, nacres, tensioning agents and matting polymers, and mixtures thereof.

The term “fillers” should be understood as meaning colorless or white, mineral or synthetic, lamellar or non-lamellar particles suitable for giving the composition body or rigidity and/or softness, a matt effect and uniformity immediately on application. Fillers that may especially be mentioned include talc, mica, alumina, silica, kaolin, Nylon® powders such as Nylon-12 (Orgasol® sold by the company Atochem), polyethylene powders, polyurethane powders, polystyrene powders, polyester powders, optionally modified starch, silicone resin microbeads such as those sold by the company Toshiba under the name Tospearl®, hydroxyapatite, hollow silica microspheres (Silica Beads® from the company Maprecos) and calcined alumina (Spectral)PC-401®).

The term “pigments” should be understood as meaning white or colored, mineral or organic particles that are insoluble in the medium, which are intended to color and/or opacify the composition. They may be of standard or nanometric size. Among the mineral pigments that may be mentioned are titanium dioxide, zirconium dioxide and cerium dioxide, and also zinc oxide, iron oxide and chromium oxide.

The term “nacres” should be understood as meaning iridescent particles that reflect light. Among the nacres that may be envisaged, mention may be made of natural mother-of-pearl, mica coated with titanium oxide, with iron oxide, with natural pigment or with bismuth oxychoride, and also colored titanium mica.

The mass concentration in the aqueous phase of these fillers and/or pigments and/or nacres is generally from 0.1% to 20% and preferably from 0.2% to 7% by weight relative to the total weight of the composition.

The term “tensioning agent” should be understood as meaning a compound suitable for making the skin taut and, by means of this tension effect, making the skin smooth and reducing or even immediately eliminating wrinkles and fine lines therefrom. Tensioning agents that may be mentioned include polymers of natural origin. The term “polymer of natural origin” means polymers of plant origin, polymers derived from integuments, egg proteins and latices of natural origin. These polymers are preferably hydrophilic. Polymers of plant origin that may especially be mentioned include proteins and protein hydrolyzates, and more particularly extracts of cereals, of legumes and of oil-yielding plants, such as extracts of corn, of rye, of wheat, of buckwheat, of sesame, of spelt, of pea, of bean, of lentil, of soybean and of lupin. The synthetic polymers are generally in the form of a latex or a pseudolatex and may be of polycondensate type or obtained by free-radical polymerization. Mention may be made especially of polyester/polyurethane and polyether/polyurethane dispersions. Preferably, the tensioning agent is a copolymer of PVP/dimethiconyl acrylate and of hydrophilic polyurethane (Aquamere S-2001® from the company Hydromer).

The term “matting polymers” means herein any polymer in solution, in dispersion or in the form of particles, which reduces the sheen of the skin and which unifies the complexion. Examples that may be mentioned include silicone elastomers; resin particles; and mixtures thereof. Examples of silicone elastomers that may be mentioned include the products sold under the name KSG® by the company Shin-Etsu, under the name Trefil®, BY29® or EPSX® by the company Dow Corning or under the name Gransil® by the company Grant Industries.

The composition used according to the invention may also comprise active agents other than those for stimulating the expression of FN3K and/or FN3K RP, and in particular at least one active agent chosen from: agents that stimulate the production of growth factors; anti-glycation or deglycating agents; agents that increase collagen synthesis or that prevent its degradation (anti-collagenase agents and especially matrix metalloprotease inhibitors); agents that increase elastic synthesis or prevent its degradation (anti-elastase agents); agents that stimulate the synthesis of integrin or of focal adhesion constituents such as tensin; agents that increase the synthesis of glycosaminoglycans or proteoglycans or that prevent their degradation (anti-proteoglycanase agents); agents that increase fibroblast proliferation; depigmenting or anti-pigmenting agents; antioxidants or free-radical scavengers or anti-pollution agents; and mixtures thereof, without this list being limiting.

Examples of such agents are especially: plant extracts and in particular extracts of Chondrus crispus, of Thermus thermophilus, of Pisum sativum (Proteasyl® TP LS), of Centella asiatica, of Scenedesmus, of Moringa pterygosperma, of witch hazel, of Castanea sativa, of Hibiscus sabdriffa, of Polianthes tuberosa, of Argania spinosa, of Aloe vera, of Narcissus tarzetta, or of liquorice; an essential oil of Citrus aurantium (Neroli); α-hydroxy acids such as glycolic acid, lactic acid and citric acid, and esters thereof; β-hydroxy acids such as salicylic acid and derivatives thereof; plant protein hydrolyzates (especially of soybean or of hazelnut); acylated oligopeptides (sold especially by the company Sederma under the trade names Maxilip®, Matrixyl® 3000, Biopeptide® CL or Biopeptide® EL or by Lipotec under the trade name Eyeseryl®); yeast extracts and in particular of Saccharomyces cerevisiae; algal extracts and in particular of laminairia; vitamins and derivatives thereof such as retinyl palmitate, ascorbic acid, ascorbyl glucoside, magnesium or sodium ascorbyl phosphate, ascorbyl palmitate, ascorbyl tetraisopalmitate, ascorbyl sorbate, tocopherol, tocopheryl acetate and tocopheryl sorbate; arbutin; kojic acid; ellagic acid; tranexamic acid or its derivatives, such as tranexamic cetyl ester; and mixtures thereof.

According to one preferred embodiment, the composition used in the process according to the invention contains at least one active agent that stimulates the synthesis of extracellular macromolecules and in particular of collagen IV and/or of hyaluronane and/or of fibronectin, such as at least one acylated oligopeptide. The oligopeptide may especially be chosen from lysyl-threonyl-threonyl-lysyl-serine, glycyl-histidyl-lysine and glycyl-glutamyl-prolyl-arginine sequences, and mixtures thereof, modified with an alkanoyl group containing at least 6 and preferably at least 10 carbon atoms, in particular a palmitoyl group. An active agent of this type is especially sold by the company Sederma under the trade name Matrixyl® 3000.

As a variant or in addition, the compound used according to the invention may comprise at least one antioxidant, which is preferably water-soluble, preferably having the property of reducing hydroperoxides, such as carcinine hydrochloride, which is sold especially by the company Exsymol under the trade name Alistin®.

As a variant or in addition, the composition used according to the invention may also comprise at least one inhibitor of a matrix metalloprotease of MMP1, MMP2, MMP3 and/or MMP9 type such as the essential oil of Citrus aurantium amara that is sold especially by the company Biolandes under the trade name Neroli Morocco Oil®.

As a variant or in addition, the composition used according to the invention may comprise at least one elastase inhibitor (anti-elastase), such as an extract of Pisum sativum seeds that is sold especially by the company Laboratoires Sérobiologiques/Cognis France under the trade name Proteasyl TP LS®.

Alternatively or additional the composition used according to the invention may comprise at least one agent that activates the microcirculation and has anti-edematous properties, such as “acetyl tetrapeptide-5”, which allows for the reduction of eye puffs and is marketed by Lipotec under the trade name Eyeseryl®; or such as escin, which is sold by Indena under the trade name Phytosome Escine/β-sitosterol®.

The invention will now be illustrated by the non-limiting examples that follow.

### EXAMPLES

### Example 1

### Preparation of an Extract of Butea frondosa

1) Aqueous-Alcoholic Extraction

1.110 kg of Butea frondosa flowers are ground using a knife mill (Retsch) and loaded into a 20 l glass reactor equipped with a reflux condenser.

7.8 l of 96% (volume/volume) ethanol are added.

Heating of the reactor is started at 50° C. Heating is continued for 5 hours.

The material is then filtered so as to remove the ground material of Butea frondosa flowers. The filtrate is recovered.

The solvent is then evaporated off on a rotary evaporator under vacuum.

0.183 kg of extract of Butea frondosa flowers is thus recovered.

The yield for this operation is 16.5%.

2) Decolorization of the Extract

The oleoresin is hot-washed with 96% (volume/volume) ethanol and active charcoal:

183 g of oleoresin are mixed with 1500 ml of 96% ethanol and 24 g of active charcoal. The mixture is stirred vigorously for 2 hours at 50-60° C. and is then left to stand at room temperature for 2 hours. After filtering the solution through a Büchner funnel, the primary filtrate is recovered.

This filtrate is then filtered again on a conical filter in order to remove the final residues of active charcoal, and the ethanol is then evaporated off using a rotary evaporator under vacuum.

The yield for this decolorization operation is 68%.

The total yield for the process is 11.2%.

### Example 2

### Test of Stimulation of the Expression of FN3K mRNA

### Example 2A

### Test on Keratinocytes

Protocol:

The effect of the botanical extract of Example 1 on the expression of the mRNA of FN3K and/or FN3K RP was evaluated on keratinocytes.

Keratinocytes derived from neonatal foreskins (Clonetics, San Diego, USA) were inoculated in 6-well plates and cultured in keratinocyte growth culture medium (KBM, Clonetics), i.e. a modified culture medium supplemented with human recombinant EGF, insulin, hydrocortisone, bovine pituitary extract, gentamycin and amphotericin B. After culturing for 24 hours in an oven at 37° C., the confluent cells were washed with PBS buffer (Gibco) and incubated with specific basic medium (KBM, Clonetics) containing the extract to be tested, for 24 hours, at increasing concentrations. After studying the cytotoxicity of the extract, its activity was evaluated.

To quantify the expression of the messenger RNA of FN3K and of FN3K RP in a treated sample relative to an untreated sample, real-time polymerase chain amplification (RT-PCR) was used. The results were normalized relative to the expression of domestic genes of these samples and corrected as regards the differences in efficacy of PCR. The results were expressed in terms of the number of times of increase or of decrease of expression of the target gene FN3K or FN3K RP in the treated sample, rather than as an absolute number of copies.

The cDNA/mRNA sequences of the genes investigated were obtained from Genbank.

Domestic Gene: PBGD

All the PCR primers were obtained using the scientific publication of Conner, J., et al., 2005. Ann. N.Y. Acad. Sci. 1043: 824:836. The keratinocytes were treated with various concentrations of extracts in triplicate for 24 hours. The mRNA was isolated using the reagent Qiagen RNeasy kit and quantified using the QuantIt kit (Invitrogen, CA).

Reverse transcription was performed using the gene Amp RNA PCR kit (Applied Biosystems, CA) according to the manufacturer's recommendations.

The real-time PCR measurement was performed using the iCYCLER IQ machine (Bio-Rad, CA) with SYBR Green I detection.

In all the tests, the cDNA was amplified using a standardized program. Each sample was charged with supermix IQ SYBR Green I, water and primer (stock). The final amount of cDNA per reaction corresponded to 75 ng of total RNA used for the reverse transcription.

The relative quantification of the expression of the target gene was performed using the Pfaffl mathematical model (Pfaffl, NW, Nucleic Acids Res. 29(9), p. E45, 2001).

The positive results were confirmed using cells from two different donors.

Results:

The results are given in Tables 1 and 2 below:

TABLE 1

| | | Stimulation of | Standard |
|---|---|---|---|
| | Concentration⁽¹⁾ | FN3K mRNA | deviation |
| Untreated | — | 1.05 | 0.01 |
| keratinocytes | | | |
| Butea frondosa | 0.02% | 1.26 | 0.07 |
| | 0.1% | 7.36 | 0.75 |

TABLE 2

| | Stimulation of | Standard |
|---|---|---|
| Active agent tested | FN3K RP mRNA | deviation |
| Untreated | 1.04 | 0.01 |
| keratinocytes | | |
| Butea frondosa at 0.1%⁽¹⁾ | 2.23 | 0.085 |

⁽¹⁾the concentrations of the extracts are expressed as the weight of crude extract per weight of preparation

It emerges from this test that the Butea frondosa extracts make it possible to stimulate the expression of the mRNA of FN3K and of FN3K RP in normal keratinocytes.

### Example 2B

### Test on Fibroblasts

Protocol:

A test similar to that of Example 2A was performed on a culture of normal human fibroblasts (Invitrogen, CA) cultured for 24 hours at 37° C. in the presence of a DMEM medium (Gibco) containing 10% fetal bovine serum (Invitrogen) and 1% penicillin-streptavidin (Invitrogen), and then washed with HBSS (Gibco). The fibroblasts were treated in triplicate with the extract of Example 1 at 0.02% by weight during 24 hours, in the presence of a medium containing 1% penicillin-streptavidin (Cellgro, CA).

Results:

The Butea frondosa extract at 0.02% was observed to stimulate the expression of FN3K in fibroblasts.

### Example 3

### Assessment of the Expression of FN3K with Age

Protocol:

The variation in expression of the FN3K protein was evaluated by immunohistochemistry (IHC), using freezed skin samples from 3 to 5 donors of various ages. Staining was performed on cryosections of 5 μm from 2 age groups (30-40 years old and 60-70 years old), with anti-FN3K antibodies (Santa Cruz, Calif.) and secondary antibodies (Jackson Immunoreasearch Labs, PA). The extent of staining was assessed on 6 sections from each donor, and a visual assessment of the sections was made using a scale from 0.5 to 5 in absolute value.

Results:

Evaluation of FN3K staining in young skins was 4.67 (±0.33) and that in elder skins was 1.83 (±0.66). This demonstrates that the amount of FN3K diminished with increasing age.

### Example 4

### Study of Epidermal Thickness of Reconstructed Skins without FN3K (Silenced FN3K)

Protocol:

Reconstructed epidermal skins were produced from human keratinocytes that were normal or transfected with two different FN3K siRNAs and an experimental control (scramble siRNA). After 6 days of culture, the reconstructed skins were stained with H&E (hematoxyline and cosine) to assess the morphology of the reconstructed skins. Epidermal thickness was then measured. For each point, 150 measurements were performed of three different skins prepared using keratinocytes from two different donors.

Results:

Silencing of the FN3K siARN results in a reduction in epidermal thickness (from 408.8 μm to 300.8 μm or 348.3 μm depending on the siARN used). This revealed a reduction in reconstructed skin growth and viability. FN3K is thus an essential element in the formation of an epidermis, whose thickness is known to decrease with age.

TABLE 3
Epidermis thickness in μm

| | SiARN1/ | siARN2/ | Experimental |
|---|---|---|---|
| Control/Standard | Standard | Standard | control/Standard |
| deviation | deviation | deviation | deviation |
| 408.8 +/− 137.46 | 300.8 +/− 77.34 | 348.3 +/− 97 | 489.1 +/− 157.7 |

### Example 5

### Expression of Catalase in FN3K-Silenced Reconstructed Skins, Compared with Glycation-Induced Reconstructed Skins

Protocol:

Glycation was induced in cultured reconstructed skins by adding 250 μg of methylglyoxal. The effects of glycation and FN3K-silencing on catalase expression in reconstructed skins were assessed by immunohistochemistry.

Results:

Catalase expression significantly decreases in glycated reconstructed skins and in FN3K-silenced skins. This demonstrates the importance of FN3K in the normal functioning of the epidermis and its protective effect against radicals.

### Example 6

### Effect of Butea frondosa on the Glycation of the Extra-Cellular Matrix (ECM) and on the Production of Essential Fatty Acids (EFA) by Fibroblasts

Protocol:

Human normal fibroblasts were cultured at 37° C. in the presence of DMEM (Invitrogen) containing 10% of fetal bovine serum (Invitrogen) and 50 UI/ml of penicillin, 50 μg/ml of streptavidin (Invitrogen) and 2 mM of glutamin. Collagen synthesis was stimulated by culturing cells in the presence of vitamin C at 20 μg/ml (Sigma) for 5 days. The cells were then washed with PBS and lysed by successive cycles of freezing and thawing.

Except for the controls, cell layers had previously been treated with Butea frondosa (at 0.02; 0.1 and 0.5%), which was prepared according to Example 1 or with a reference compound: aminoguanidin at 1 mg/ml (Sigma). The cells were then incubated in the presence of 0.1 mg/ml glucose. Each of the treatments was performed, in triplicate, for 15 days at 37° C. with 5% CO2.

At the end of the incubation period, ECM proteins (essentially collagen) were extracted, and the samples were then transferred onto a nitrocellulose membrane (Hybrond, ECL, Amersham) by means of a Milliblot dotblot (Millipore). All the controls were carried out in order to assess the specificity of the final response. No interference was observed.

The non-specific sites of the membranes were then saturated for a whole night at 4° C. in a saturation medium made of a phosphate buffer (PBS) containing 0.05% of Tween 20 and 5% of delipidated milk (PBS™). After several washing cycles in the PBS™ medium, the specific antigen sites were stained using an anti-EFA monoclonal antibody (Euromedex) diluted in PBS™, then revealed using an anti-mouse immunoglobulin-peroxidase (Sigma, 1/200 in PBS™, 1 hour at 37° C.). Following several washing cycles with PBS™, the peroxidase activity was revealed by means of a chemiluminescence method (ECL, Amersham) on a Kodak MR film.

Results:

The following table illustrates the effect of this treatment on glucose incorporation into macromolecules synthetised by fibroblasts. The presence of EFA was measured by dot blot analysis and densitometric analysis.

TABLE 4

| | |
|---|---|
| | Butea Frondosa extract |
| Aminoguanidin | according to Example 1 |

| Control | 1 mg/ml | 0.02% | 0.1% | 0.5% |
|---|---|---|---|---|
| 100% | 65% | 77% | 89% | 65% |

The aminoguanidin reference compound, tested at 1 mg/ml, decreases significantly the EFA content (35% inhibition compared to the control). This result validates the test. Butea frondosa tested at 0.02; 0.1 and 0.5% also also found to reduce the EFA content (from 11% to 35% inhibition compared to the control).

Therefore, the Butea frondosa extract reduces the non-enzymatic glycation of the ECM.

### Example 7

### Cosmetic Composition

The following compositions may be prepared in a manner that is conventional for those skilled in the art. The amounts indicated below are expressed as weight percentages. The ingredients in upper case are identified in accordance with the INCI name.

7A - Night cream

| | |
|---|---|
| Tetrasodium EDTA | 0.05% |
| GLYCERYL POLYMETHACRYLATE & PROPYLENE | 5.00% |
| GLYCOL⁽¹⁾ | |
| Cetearyl alcohol | 2.36% |
| Glycerol | 8.36% |
| Glycols | 5.27% |
| Aqueous-phase gelling agents | 0.57% |
| Sodium hyaluronate | 0.05% |
| Nonionic emulsifiers | 2.64% |
| Emollients | 19.00% |
| Extract of Citrus aurantium blossom⁽²⁾ | 0.0025% |
| Extract of Pisum sativum⁽³⁾ | 5.00% |
| Water-glycol solution of | 1.00% |
| decarboxycarnosine hydrochloride⁽⁴⁾ | |
| Film-forming polymers | 1.00% |
| PALMITOYL OLIGOPEPTIDE - PALMITOYL | 3.00% |
| TETRAPEPTIDE-3⁽⁵⁾ | |
| Fillers | 1.00% |
| Preserving agents | 0.60% |
| Extract of Butea frondosa⁽⁶⁾ | 0.05% |
| Fragrance | qs |
| Dyes | qs |
| Water | qsp 100.00% |

⁽¹⁾Lubrajel MS ® from Guardian Laboratories
⁽²⁾Neroli Morocco Oil from Biolandes
⁽³⁾Proteasyl ® TP LS 8657 from Cognis
⁽⁴⁾Alistin ® from Exsymol
⁽⁵⁾Matrixyl ® 3000 from Sederma
⁽⁶⁾as described in Example 1 and then diluted to 80% by weight in dipropylene glycol

7B - SPF 15 fluid

| | |
|---|---|
| UV-screening agents | 10.50% |
| Cetyl alcohol | 1.00% |
| Emollients | 12.00% |
| Nonionic emulsifier | 4.50% |
| Tetrasodium EDTA | 0.10% |
| Glycerol | 8.70% |
| Glycols | 2.93% |
| Aqueous-phase gelling agents | 0.95% |
| Sodium hyaluronate | 0.03% |
| Aqueous mixtures of acylated | 3.00% |
| oligopeptides⁽¹⁾ | |
| Extract of Pisum sativum⁽²⁾ | 5.00% |
| Water-glycol solution of | 1.00% |
| decarboxycarnosine hydrochloride⁽³⁾ | |
| Fillers | 6.00% |
| Film-forming polymer | 0.40% |
| Preserving agents | 0.51% |
| Extract of Butea frondosa⁽⁴⁾ | 0.05% |
| Fragrance | qs |
| Dyes | qs |
| Water | qsp 100.00% |

⁽¹⁾Matrixyl ® 3000 from Sederma
⁽²⁾Proteasyl ® TP LS 8657 from Cognis
⁽³⁾Alistin ® from Exsymol
⁽⁴⁾as described in Example 1 and then diluted to 80% by weight in dipropylene glycol

7C - Serum

| | |
|---|---|
| Glycerol | 9.75% |
| Glycol | 5.94% |
| Solvent | 10.00% |
| Emollient | 5.00% |
| Fillers | 3.15% |
| Film-forming polymer | 0.40% |
| Aqueous-phase gelling agents | 1.00% |
| Tetrasodium EDTA | 0.05% |
| Extract of Citrus aurantium blossom⁽¹⁾ | 0.01% |
| Aqueous mixtures of acylated | 3.00% |
| oligopeptides⁽²⁾ | |
| Extract of Pisum sativum⁽³⁾ | 4.00% |
| Water-glycol solution of | 1.00% |
| decarboxycarnosine hydrochloride⁽⁴⁾ | |
| Extract of Butea frondosa⁽⁵⁾ | 0.05% |
| Fragrance | qs |
| Dyes | qs |
| Water | qsp 100.00% |

⁽¹⁾Neroli Morocco Oil from Biolandes
⁽²⁾Matrixyl ® 3000 from Sederma
⁽³⁾Proteasyl ® TP LS 8657 from Cognis
⁽⁴⁾Alistin ® from Exsymol
⁽⁵⁾as described in Example 1 and then diluted to 80% by weight in dipropylene glycol

7D - SPF 15 day cream

| | |
|---|---|
| UV filter | 10.99% |
| Tetrasodium EDTA | 0.10% |
| Emollients | 8.00% |
| Cetyl alcohol | 2.04% |
| Glycerol | 5.86% |
| Glycols | 1.81% |
| Humectants | 4.52% |
| Aqueous-phase gelling agents | 1.02% |
| Nonionic emulsifiers | 3.96% |
| Extract of Citrus aurantium blossom⁽¹⁾ | 0.0025% |
| Extract of Pisum sativum⁽¹⁾ | 5.00% |
| Water-glycol solution of | 1.00% |
| decarboxycarnosine hydrochloride⁽³⁾ | |
| Film-forming polymers | 2.00% |
| PALMITOYL OLIGOPEPTIDE - PALMITOYL | 3.00% |
| TETRAPEPTIDE-3⁽⁴⁾ | |
| Fillers | 6.00% |
| Preserving agents | 1.18% |
| pH-adjuster | 0.15% |
| Extract of Butea frondosa⁽⁵⁾ | 0.05% |
| Fragrance | qs |
| Dyes | qs |
| Water | qsp 100.00% |

⁽¹⁾Neroli Morocco Oil from Biolandes
⁽²⁾Proteasyl ® TP LS 8657 from Cognis
⁽³⁾Alistin ® from Exsymol
⁽⁴⁾Matrixyl ® 3000 from Sederma
⁽⁵⁾as described in Example 1 and then diluted to 80% by weight in dipropylene glycol

7E - Eye cream

| | |
|---|---|
| Tetrasodium EDTA | 0.10% |
| Emollients | 7.70% |
| Volatile silicones | 10.90% |
| Cetyl alcohol | 1.02% |
| Glycerol | 8.70% |
| Glycols | 6.20% |
| Sodium hyaluronate | 0.03% |
| Aqueous-phase gelling agents | 1.11% |
| Nonionic emulsifiers | 3.98% |
| Extract of Citrus aurantium blossom⁽¹⁾ | 0.0025% |
| Extract of Pisum sativum⁽²⁾ | 5.00% |
| Water-glycol solution of | 1.00% |
| decarboxycarnosine hydrochloride⁽³⁾ | |
| Film-forming polymers | 2.00% |
| PALMITOYL OLIGOPEPTIDE - PALMITOYL | 3.00% |
| TETRAPEPTIDE-3⁽⁴⁾ | |
| Extract of Butea frondosa⁽⁵⁾ | 0.05% |
| ACETYL TETRAPEPTIDE-5⁽⁶⁾ | 1.00% |
| Escin⁽⁷⁾ | 0.40% |
| Fillers | 5.55% |
| Preserving agents | 0.61% |
| pH adjuster | 0.15% |
| Fragrance | qs |
| Dyes | qs |
| Water | qsp 100.00% |

⁽¹⁾Neroli Morocco Oil from Biolandes
⁽²⁾Proteasyl ® TP LS 8657 from Cognis
⁽³⁾Alistin ® from Exsymol
⁽⁴⁾Matrixyl ® 3000 from Sederma
⁽⁵⁾as described in Example 1 and then diluted to 80% by weight in dipropylene glycol
⁽⁶⁾Eyeseryl from Lipotec
⁽⁷⁾Escin Phytosome from INDENA

These compositions may be applied daily, morning and/or evening, to facial skin to make it supple, smooth and luminous and to combat wrinkles and slackening of the skin.

1-7. (canceled)

8. Cosmetic process for preventing and/or combating at least one sign of ageing of the skin, comprising the topical application to the skin of a composition containing at least one active agent that stimulates the expression of fructosamine-3-kinaase (FN3K) and/or its related protein (FN3K RP).

9. Process according to claim 8, wherein the sign of ageing of the skin is chosen from: the formation of wrinkles and fine lines and/or loss of firmness and/or loss of elasticity of the skin.

10. Cosmetic process for preventing and/or combating the “orange peel” appearance of the skin, comprising the topical application to the skin of a composition containing at least one active agent that stimulates the expression of FN3K and/or FN3K RP.

11. Process according to claim 8, wherein the active agent that stimulates the expression of FN3K and/or FN3K RP is a botanical extract.

12. Process according to claim 11, wherein the active agent is an alcoholic extract of Butea frondosa blossom.

13. Process according to claim 12, wherein the extract is obtained by extraction using at least one monoalcohol and/or at least one glycol, optionally mixed with water.

14. Process according to claim 10, wherein the active agent that stimulates the expression of FN3K and/or FN3K RP is a botanical extract.

15. Process according to claim 14, wherein the active agent is an alcoholic extract of Butea frondosa blossom.

16. Process according to claim 15, wherein the extract is obtained by extraction using at least one monoalcohol and/or at least one glycol, optionally mixed with water.
